(19) United States
(12) Patent Application Publication
(10) Pub. No.: US 2025/0057998 A1
(43) Pub. Date: Feb. 20, 2025

(54) Radiopharmaceutical and Methods

(71) Applicant: POINT BIOPHARMA, INC., Indianapolis, IN (US)

(72) Inventors: Joe McCANN, Toronto (CA); Mark DORNAN, Toronto (CA)

(21) Appl. No.: 18/817,480

(22) Filed: Aug. 28, 2024

Related U.S. Application Data
(63) Continuation of application No. PCT/CA2023/050269, filed on Mar. 2, 2023.
(60) Provisional application No. 63/316,381, filed on Mar. 3, 2022.
(60) Provisional application No. 63/315,974, filed on Mar. 2, 2022.

(51) Int. Cl. A61K 51/08; A61K 47/22; A61K 51/12

(52) U.S. Cl. CPC A61K 51/083; A61K 47/22; A61K 51/088; A61K 51/121

(57) ABSTRACT

A radiopharmaceutical ¹⁷⁷Lu-DOTATATE compound, a composition, and a kit are provided. Further provided are methods of synthesis of a ¹⁷⁷Lu-DOTATATE compound and methods of treatment that comprise a ¹⁷⁷Lu-DOTATATE compound.

### RELATED APPLICATIONS

This application is a continuation of International Application No. PCT/CA2023/050269, which designated the United States and was filed on Mar. 2, 2023, published in English, which claims the benefit of U.S. Provisional Application No. 63/315,974 filed Mar. 2, 2022 and U.S. Provisional Application No. 63/316,381 filed Mar. 3, 2022. The entire contents of the above-referenced applications are incorporated by reference herein.

### FIELD OF THE INVENTION

The radiopharmaceutical ¹⁷⁷Lu-DOTATATE (¹⁷⁷Lu-octreotate) and a pharmaceutical composition thereof are provided. Further provided are methods of preparing ¹⁷⁷Lu-DOTATATE compound and pharmaceutical compositions that include ¹⁷⁷Lu-DOTATATE.

### BACKGROUND OF THE INVENTION

Radiopharmaceuticals have been used for a variety of therapeutic and diagnostic indications. Among others, radiolabeled molecules have been useful to treat various malignant tumors.

Use of these agents presents certain challenges, including with respect to stability and shelf-life. In particular, therapeutic compositions comprising a radionuclide may undergo radiolysis during preparation and storage. During radiolysis, radionuclide emissions may react with other groups of the pharmaceutical agent thereby resulting in decomposition of the agent as well as undesired effects.

It thus would be desirable to have additional and improved radiopharmaceutical agents.

### SUMMARY OF THE INVENTION

We now provide, inter alia, radiopharmaceutical ¹⁷⁷Lu-DOTATATE compound, methods of preparing the compound, and pharmaceutical compositions and methods of treatment including ¹⁷⁷Lu-DOTATATE.

We have now found, inter alia, 1) new methods to produce high purity ¹⁷⁷Lu-DOTATATE including with high levels of ¹⁷⁷Lu incorporation, mild reaction temperatures and/or reduced reaction times and 2) new ¹⁷⁷Lu-DOTATATE pharmaceutical compositions that maintain radiochemical purity for extended storage times following preparation (e.g. >90 or 95%, 3, 4 or 5 days or more, 25° C.).

The preferred methods and pharmaceutical compositions include one or more ascorbate compounds such as ascorbic acid, but do not contain other stabilizer agents. In particular aspects, the present methods and pharmaceutical compositions are substantially or completely free of a gentisate compound such as gentisic acid.

As referred to herein, one or more gentisate compounds would be substantially absent (or an admixture comprising lutetium-177 and EuK-Sub-kf-iodo-y-DOTAGA would be substantially free of gentisate compound(s)) if one or more gentisate compounds are present in an amount of less than 10, 8, 5, 4, 3, 2, 1, 0.5, 0.25, or 0.1 weight percent relative to the weight amount of one or more ascorbate compounds. In certain aspects, a gentisate compound such as gentisic acid can be completely absent (e.g. effectively undetectable (e.g. by HPLC or other analysis) including by such analysis zero weight percent relative to the weight amount of one or more ascorbate compounds).

More particularly, in one aspect, pharmaceutical compositions are provided that comprise: (a) a complex of lutetium-177 and tetraazacyclododecane tetra-acetic acid-octreotate (DOTATATE); and (b) one or more or more ascorbate compounds, in the substantial or complete absence of a gentisate compound such as gentisic acid. Such pharmaceutical composition may comprise one or more other components such as one or more sequestering agents, an organic or inorganic base, salt and/or buffering agents.

In a further aspect, pharmaceutical compositions are provided that comprise: (a) a complex of lutetium-177 and tetraazacyclododecane tetra-acetic acid-octreotate (DOTATATE); and (b) one or more or more ascorbate compounds such as ascorbic acid, in the substantial or complete absence of a gentisate compound (e.g. such as gentisic acid) or additional stabilizer compounds other than an ascorbate compound. Such pharmaceutical composition may comprise one or more other components such as one or more sequestering agents, organic or inorganic base, a salt such as NaCl, and/or one or more buffering agents.

In one preferred aspect, pharmaceutical compositions are provided that consist essentially of: (a) a complex of lutetium-177 and tetraazacyclododecane tetra-acetic acid-octreotate (DOTATATE); and (b) one or more or more ascorbate compounds such as ascorbic acid, in the substantial or complete absence of a gentisate compound such as gentisic acid. Such pharmaceutical composition may comprise one or more other components such as one or more sequestering agents, organic or inorganic base, a salt such as NaCl, and/or one or more buffering agents.

In certain aspects, suitably, the complex of lutetium-177 and tetraazacyclododecane tetra-acetic acid-octreotate (DOTATATE) is present in an amount of at least 5, 10, 15, 20, 25 or 30 mCi/mL.

In certain preferred aspects, one or more ascorbate compounds such as ascorbic acid are present in a pharmaceutical composition in an amount of 1 mg/mL or greater.

In certain preferred aspects, one or more ascorbate compounds such as ascorbic acid are present in a pharmaceutical composition in an amount of 2 mg/mL or greater.

In certain preferred aspects, one or more ascorbate compounds such as ascorbic acid are present in a pharmaceutical composition in an amount of 3 mg/mL or greater.

In certain preferred aspects, one or more ascorbate compounds such as ascorbic acid are present in a pharmaceutical composition in an amount of 4 mg/mL or greater.

In certain preferred aspects, one or more ascorbate compounds such as ascorbic acid are present in a pharmaceutical composition in an amount of 5 mg/mL or greater.

In certain preferred aspects, one or more ascorbate compounds such as ascorbic acid are present in a pharmaceutical composition in an amount of 6 mg/mL or greater.

In certain preferred aspects, one or more ascorbate compounds such as ascorbic acid are present in a pharmaceutical composition in an amount of 7 mg/mL or greater.

In certain preferred aspects, one or more ascorbate compounds such as ascorbic acid are present in a pharmaceutical composition in an amount of 8 mg/mL or greater.

In certain preferred aspects, one or more ascorbate compounds such as ascorbic acid are present in a pharmaceutical composition in an amount of 9, 10, 11, 12, 13, 14, 15, 16, 17, 18, 19 or 20 mg/mL or greater.

In certain preferred aspects, one or more ascorbate compounds such as ascorbic acid are present in a pharmaceutical composition in an amount of not exceeding 20, 19, 18, 17, 16, 15, 14, 13, 12, 11 or 10 mg/mL.

In certain preferred aspects, one or more ascorbate compounds such as ascorbic acid are present in a pharmaceutical composition in an amount of not exceeding 20, 19, 18, 17, 16, 15, 14, 13, 12, 11 or 10 mg/mL.

In certain preferred aspects, one or more ascorbate compounds such as ascorbic acid are present in a pharmaceutical composition in an amount of between 2 and 10 mg/mL.

In certain preferred aspects, one or more ascorbate compounds such as ascorbic acid are present in a pharmaceutical composition in an amount of between 2 and 5 mg/mL.

In certain preferred aspects, one or more ascorbate compounds such as ascorbic acid are present in a pharmaceutical composition in an amount of between 3 and 5 mg/mL.

In certain preferred aspects, one or more ascorbate compounds such as ascorbic acid are present in a pharmaceutical composition in an amount of between 3 and 4 mg/mL.

In certain preferred aspects, one or more ascorbate compounds such as ascorbic acid are present in a pharmaceutical composition in an amount of 3.3 mg/mL, 3.4 mg/mL. 3.5 mg/mL, 3.6 mg/mL, 3.7 mg/mL or 3.8 mg/mL.

In certain aspects, a pharmaceutical composition will contain a single ascorbate compound. In certain additional preferred aspects, a pharmaceutical composition will contain a single ascorbate compound that is ascorbic acid.

It has been found that a substantial or complete absence of one or more gentisate compounds during the incorporation reaction can reduce or avoid the occurrence or formation of an impurity that has been detected by high-performance liquid chromatography (HPLC).

In certain aspects, pharmaceutical composition will be substantially or completely free of a gentisate adduct impurity. An impurity or impurities observed upon use of one or more gentisate compounds such as gentisic acid for example during the incorporation reaction to form a complex of lutetium-177 and tetraazacyclododecane tetra-acetic acid-octreotate (DOTATATE) or otherwise may be present in an incorporation reaction admixture or pharmaceutical composition is sometimes referred to herein as “gentisate adduct impurity”. A gentisate adduct impurity can be assessed by HPLC. A pharmaceutical composition will be substantially free of a gentisate adduct impurity if an HPLC analysis shows minimal or no observation (naked eye observation of HPLC chromatograph) of the gentisate adduct impurity. A pharmaceutical composition will be completely free of a gentisate adduct impurity if an HPLC analysis shows no observation (naked eye observation of HPLC chromatograph) of the gentisate adduct impurity.

Suitably, the pharmaceutical composition is an aqueous formulation. In certain preferred aspects, a pharmaceutical composition does not contain an alcohol such as ethanol or other organic solvent. In certain preferred aspects, a pharmaceutical composition is at least substantially free (i.e. less than 5, 4, 3, 2, 1 or 0.5 weight percent based on total composition weight) of an alcohol such as ethanol or other organic solvent.

In certain preferred aspects, a pharmaceutical composition has a pH of 4.0 to 6.5. In additional preferred aspect, pharmaceutical composition has a pH of 4.5 to 6.0.

The compound tetraazacyclododecane tetra-acetic acid-octreotate (DOTATATE) has the following structure 1:

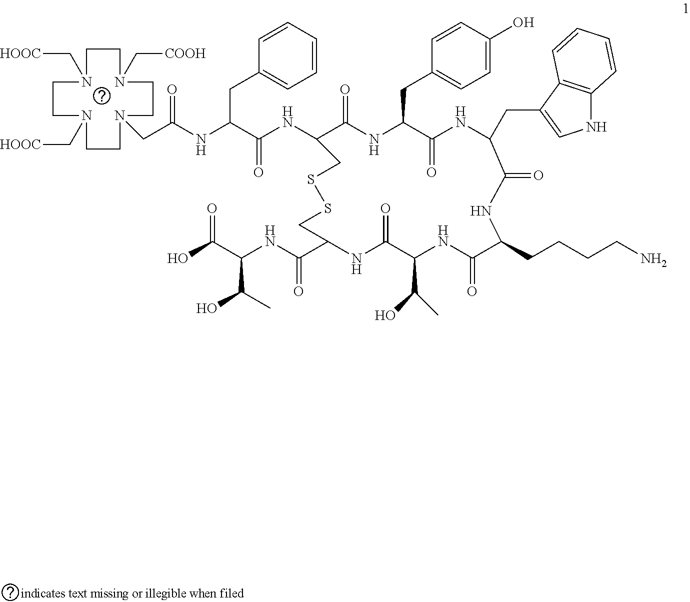

¹⁷⁷Lu-DOTATATE is a lutetium-177 complex of the above compound 1 and may be represented by the following structure 2:

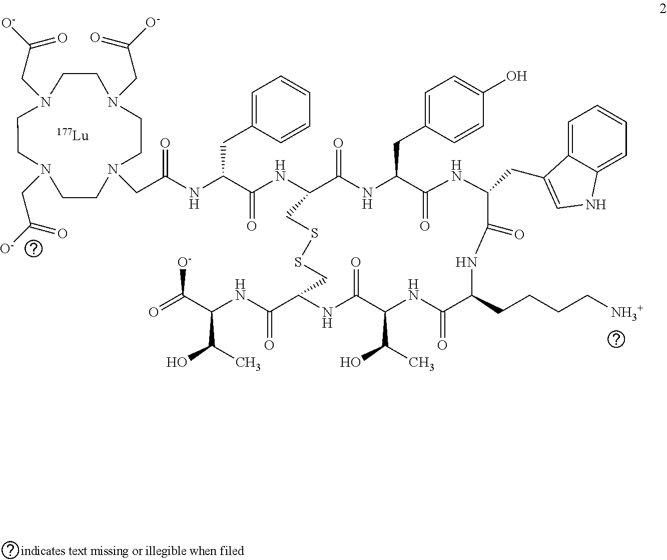

In an aspect, methods are provided for preparing ¹⁷⁷Lu-DOTATATE (structure 2 above), which includes admixing lutetium-177 and tetraazacyclododecane tetra-acetic acid-octreotate (DOTATATE, structure 1 above) in the presence of one or more ascorbate compounds and forming a complex of ¹⁷⁷Lu and DOTATATE. This reaction to form a complex of ¹⁷⁷Lu and DOTATATE (e.g. such complex including structure 2 above) that may include admixing ¹⁷⁷Lu and DOTATATE is sometimes referred to herein as an “incorporation reaction”.

The one or more ascorbate compounds are suitably present in an incorporation reaction and/or a pharmaceutical composition in an amount of 20 g/mL or less, including 18, 16, 14, 12, 0, 9, 8, 7, 6, 5, 4, 3 or 2 mg/mL or less.

In preferred aspects, the tetraazacyclododecane tetra-acetic acid-octreotate (DOTATATE) is admixed with lutetium-177 as an aqueous formulation in the incorporation reaction mixture.

In certain aspects, in an incorporation reaction and/or a pharmaceutical composition, the one or more ascorbate compounds include ascorbic acid. In certain aspects, in an incorporation reaction and/or a pharmaceutical composition, the one or more ascorbate compounds include an ascorbate salt such as sodium ascorbate.

Preferably, prior to the incorporation reaction or addition to ¹⁷⁷Lu-DOTATATE to provide a pharmaceutical composition, the ascorbate compound such as sodium ascorbate or ascorbic acid is assessed for purity or absence of a material that may inhibit the incorporation reaction of lutetium-177 and tetraazacyclododecane tetra-acetic acid-octreotate (DOTATATE). For instance, a sample from a lot of an ascorbate compound such as an ascorbate metal salt e.g. sodium ascorbate can be tested such as in a test-scale (small-scale) incorporation reaction of lutetium-177 and tetraazacyclododecane tetra-acetic acid-octreotate (DOTATATE) to ensure the ascorbate compound lot does not adversely impact the formation of the complex of lutetium-177 and tetraazacyclododecane tetra-acetic acid-octrotate (DOTATATE) or the radiochemical stability of the formed complex over time.

Accordingly, methods are provided that comprise determining purity of an ascorbate compound prior to admixing the ascorbate compound with lutetium-177 and/or tetraazacyclododecane tetra-acetic acid-octreotate (DOTATATE) and/or a complex of lutetium-177 and/or tetraazacyclododecane tetra-acetic acid-octreotate (DOTATATE). Suitably, the ascorbate compound is determined to be effective in an incorporation reaction and method comprises admixing the effective ascorbate compound with lutetium-177 and/or tetraazacyclododecane tetra-acetic acid-octreotate (DOTATATE) and/or a complex of lutetium-177 and/or tetraazacyclododecane tetra-acetic acid-octreotate (DOTATATE). In such methods, the ascorbate compound may be ascorbic acid or an ascorbate salt, such as sodium ascorbate.

In certain preferred aspects, the admixture of lutetium-177 and tetraazacyclododecane tetra-acetic acid-octreotate (DOTATATE) is heated. In particular aspects, the admixture is heated for 60 minutes or less, for 50 minutes or less, for 40 minutes, for 30 minutes or less, for 20 minutes or less, or for 15 minutes or less.

In certain preferred aspects, the admixture of lutetium-177 and tetraazacyclododecane tetra-acetic acid-octreotate (DOTATATE) is heated at about 80° C.±10° C., or at about 80° C.±5° C.

In certain preferred aspects, the present methods include cooling the admixture following heating.

In certain preferred aspects, following the admixing, the complex of lutetium-177 and tetraazacyclododecane tetra-acetic acid-octreotate (DOTATATE) is mixed with a formulation composition. Preferably, the formulation composition is an aqueous composition that includes one or more stabilizer compounds, particularly one or more ascorbate compounds, such ascorbic acid. The formulation composition suitably includes one or more additional stabilizer compounds present in an amount of 1, 2, 3 5, 6, 7, 8, 9, 100, 11, 12, 13, 14, 15 or 20 mg/mL or greater. As discussed above, the formulation composition is preferably at least substantially or completely free of a gentisate compound such as gentisic acid.

In certain preferred aspects, a formulation composition further comprises one or more sequestering agents.

In certain preferred aspects, incorporation of lutetium-177 into or with tetraazacyclododecane tetra-acetic acid-octreotate (DOTATATE) to provide ¹⁷⁷Lu-DOTATATE is preferably greater than 98 mole percent, or greater than 99 mole percent based on the molar equivalent of lutetium-177 used in the incorporation reaction.

In certain preferred aspects, an acidic aqueous formulation of lutetium-177 is admixed with the tetraazacyclododecane tetra-acetic acid-octreotate (DOTATATE). Preferably, a hydrogen halide or acid halide aqueous formulation of lutetium-177 is admixed with the tetraazacyclododecane tetra-acetic acid-octreotate (DOTATATE). For example, a hydrochloride acid aqueous formulation of lutetium-177 is admixed with the tetraazacyclododecane tetra-acetic acid-octreotate (DOTATATE).

In an aspect, provided is ¹⁷⁷Lu-DOTATATE obtainable, or obtained by the methods described herein.

In an aspect, provided is a pharmaceutical composition including ¹⁷⁷Lu-DOTATATE as described herein. Preferably, radiochemical purity of the composition is 95% or greater for 3 days or more at 25° C., or 95% or greater for 4 days or more at 25° C., or 95% or greater for 5 days or more at 25° C. As referred to herein, radiochemical purity is preferably assessed via HPLC analysis, particularly HPLC radiometric detection (i.e. radio-HPLC).

In an aspect, provided is a single dosage kit including ¹⁷⁷Lu-DOTATATE or pharmaceutical composition described herein. Moreover, provided is a multiple dosage kit including the ¹⁷⁷Lu-DOTATATE or pharmaceutical composition described herein.

In an aspect, provided is a method of treating a subject suffering from cancer. The method includes administering to the subject an effective amount of ¹⁷⁷Lu-DOTATATE or pharmaceutical composition described herein.

In certain aspects, the subject is suffering from a neuroendocrine tumor. In certain aspects, the subject is suffering from neuroendocrine tumors that originate from foregut, hindgut, midgut, lung, ovary, medulla, adrenal medulla, adrenal, kidney, pituitary, thyroid or paraganglia.

In certain aspects, the subject is suffering from a gastroenteropancreatic neuroendocrine tumor (GEP-NET), foregut, midgut and hindgut neuroendocrine tumors.

In certain aspects, the subject is suffering from unresectable or metastatic neuroendocrine tumor(s).

In certain aspects, the methods include identifying a patient for treatment by assessing neuroendocrine tumors of the patient to be somatostatin receptor positive, for example with ⁶⁸Ga-DOTATATE and a positron emission tomography scan.

Use of the ¹⁷⁷Lu-DOTATATE compound and compositions to treat a disease or disorder as disclosed herein is also provided.

Further provided are methods for manufacture of a medicament that comprises the ¹⁷⁷Lu-DOTATATE compound and composition to treat a disease or disorder as disclosed herein.

Other aspects of the invention are disclosed infra.

### DETAILED DESCRIPTION OF THE INVENTION

As discussed above, ¹⁷⁷Lu-DOTATATE is a lutetium-177 (¹⁷⁷Lu³⁺) complex of the compound 1

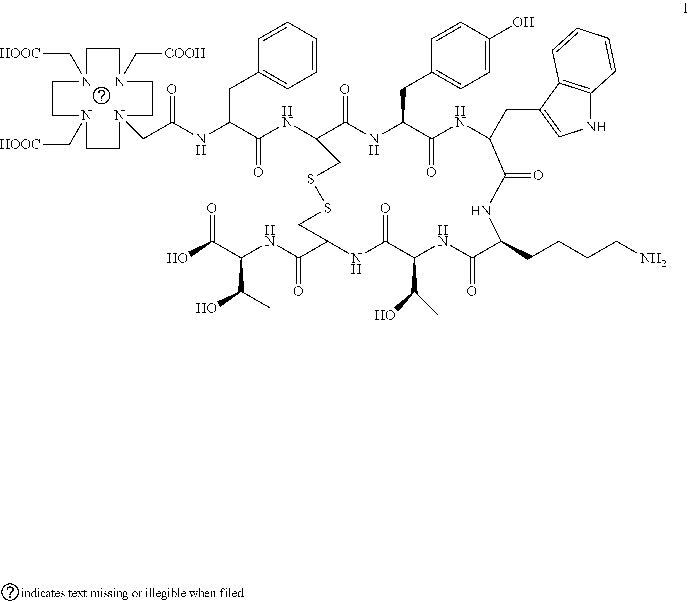

and may be represented by the following structure 2:

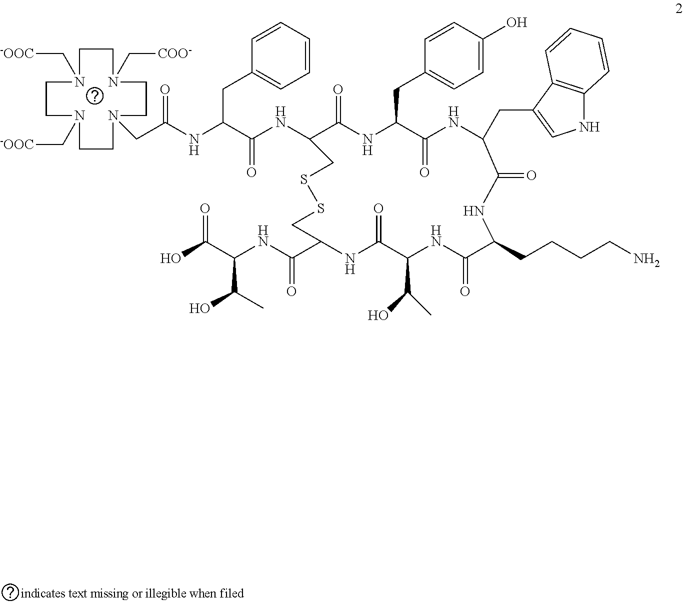

¹⁷⁷Lu-DOTATATE is also represented with a chemical (IUPAC) name of ¹⁷⁷Lu-tetraazacyclododecane tetra-acetic acid-octreotate or ¹⁷⁷Lu-4,7,10-Tricarboxymethyl-1,4,7,10-tetraaza-cyclododecan-1-yl-acetyl-D-Phe-Cys-Tyr-D-Trp-Lys-Thr-Cys-Thr-OH. ¹⁷⁷Lu-DOTATATE has a molecular formula of C₆₅H₉₀[¹⁷⁷Lu]N₁₄O₁₉S₂ and a molecular weight (average molecular weight) of 1610.61 g/mol.

### Definitions

The present invention, including compounds, methods, and pharmaceutical compositions/formulations will be described with reference to the following definitions which, for convenience, are set forth below. Unless otherwise specified, the below terms used herein are defined as follows:

As used herein, the term “a,” “an,” “the” and similar terms used in the context of the present invention (especially in the context of the claims) are to be construed to cover both the singular and plural unless otherwise indicated herein or clearly contradicted by the context.

The language “and/or” is used herein as a shorthand notation to represent the expression “and,” describing the combination of items, as well as “or,” describing the items in the alternative form.

Unless otherwise stated, structures depicted herein are also meant to include all stercochemical forms of the structure; i.e., the R and S configurations for each asymmetric center. Therefore, single stereochemical isomers as well as enantiomeric and diastereomeric mixtures of the present compounds are within the scope of the invention.

The term “about”, as used herein, means an acceptable margin of error for a particular value, which depends in part on how the value is measured or determined. In certain aspects, “about” as used herein will be understood by persons of ordinary skill in the art to mean up to plus or minus 20% of the particular term. In further aspects, “about” as used herein will be understood by persons of ordinary skill in the art to mean up to plus or minus 10% of the particular term.

As used herein, the term “substantially pure” means sufficiently homogeneous to appear free of readily detectable impurities as determined by standard analytical methods, such as thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain aspects, “substantially pure” refers to a collection of molecules, wherein at least about 55%, about 60%, about 65%, about 70%, about 75%, about 80%, about 85%, about 90%, about 95%, about 97%, about 98%, about 98.5%, about 99%, about 99.5% or about 99.9% or greater of the molecules are a single compound, including a racemic mixture or a single stereoisomer thereof, as determined by standard analytical methods.

As used herein, and unless otherwise specified, the terms “treat,” “treating” and “treatment” refer to the eradication or amelioration of a disease, disorder, or condition, or of one or more symptoms associated with the disease, disorder or condition. In certain aspects, the terms refer to minimizing the advancement or worsening of the disease, disorder, or condition resulting from the administration of a formulation of the invention to a patient with such a disease, disorder, or condition. In some aspects, the terms refer to the administration of a formulation provided herein, after the onset of symptoms of the particular disease, disorder, or condition. The terms “treat,” “treating”, “treatment”, or the like, as used herein covers the treatment of a disease, disorder, or condition in a subject, e.g., a mammal, and includes at least one of: (i) inhibiting the disease, disorder, or condition, i.e., partially or completely halting its progression; (ii) relieving the disease, disorder, or condition, i.e. causing regression of symptoms of the disease, disorder, or condition, or ameliorating a symptom of the disease, disorder, or condition; and (iii) reversal or regression of the disease, disorder, or condition, preferably eliminating or curing of the disease, disorder, or condition.

In a particular embodiment the terms “treat,” “treating”, “treatment”, or the like, covers the treatment of a disease, disorder, or condition in a mammal, e.g., a primate, e.g., a human, and includes at least one of (i), (ii), and (iii) above. As is known in the art, adjustments for age, body weight, general health, sex, diet, time of administration, drug interaction and the severity of the condition may be necessary, and will be ascertainable with routine experimentation by one of ordinary skill in the art based on the invention described herein.

As used herein, the terms “subject”, and “patient” are used interchangeably. The terms “subject” and “patient” refer to an animal such as a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

### Syntheses

In an aspect, ¹⁷⁷Lu-DOTATATE (including structure 2) can be prepared by complexing or incorporating ¹⁷⁷Lu (Lutetium 177) or halide thereof such as ¹⁷⁷LuCl₃ with DOTATATE (including structure 1 below):

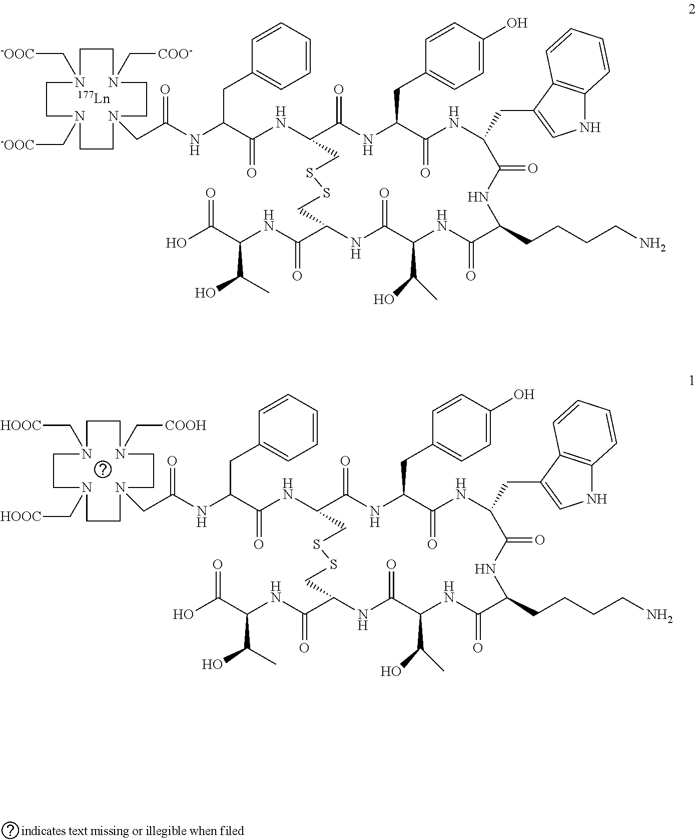

The compound 1 DOTATATE may be suitably formed as described previously such as in Raheem et al. Bioconjugate Chem. 2020. https://doi.org/10.1021/acs.bioconjchem.0c00325. The compound 1 DOTATATE is also commercially available.

It is understood that ¹⁷⁷Lu-DOTATATE as referred to herein includes the above structure 2 as well as other complexes of ¹⁷⁷Lu-DOTATATE. For instance, references herein to ¹⁷⁷Lu-DOTATATE include compounds that generally correspond to structure 2 but where the ¹⁷⁷Lu substantially complexes to other portions or moieties (such as one or more other nitrogens) of the DOTATATE molecule than as depicted in 2 above. References to ¹⁷⁷Lu-DOTATATE also may include other stereoisomers than those shown in 1 and 2 above, although the stereoisomer depicted in 1 and 2 is preferred.

To synthesize ¹⁷⁷Lu-DOTATATE, a salt form of lutetium-177 (¹⁷⁷Lu) can be admixed with DOTATATE in an incorporation reaction. The ¹⁷⁷Lu suitably may be carrier-added or more preferably no-carrier-added (n.c.a.) lutetium-177. To facilitate incorporation (e.g. complexing including chelating) of lutetium-177 with DOTATATE compound, preferably an admixture of the compounds is in the presence of one or more ascorbate compounds. In certain aspects, the incorporation reaction is substantially or completely free of other stabilizer compounds particularly a gentisate compound such as gentisic acid.

In an aspect, the method of preparing ¹⁷⁷Lu-DOTATATE includes a step of admixing lutetium-177 and tetraazacyclododecane tetra-acetic acid-octreotate (DOTATATE) in the presence of one or more ascorbate compounds such as ascorbic acid; and forming a complex of lutetium-177 and tetraazacyclododecane tetra-acetic acid-octreotate (DOTATATE). In certain aspects, the incorporation reaction is substantially or completely free of other stabilizer compounds particularly a gentisate compound such as gentisic acid.

Preferably, as discussed, in an incorporation reaction, the one or more ascorbate compounds such as ascorbic acid such as ascorbic acid are present in an amount of 1, 2, 3, 4, 5, 6, 7, 8, 9 or 10 mg/mL or greater.

Preferably, the one or more ascorbate compounds include an ascorbate salt such sodium ascorbate and/or ascorbic acid.

A gentisate compound as referred to herein includes e.g. gentisic acid.

Preferably, the admixture of lutetium-177 and tetraazacyclododecane tetra-acetic acid-octreotate (DOTATATE) is heated. In certain aspects, the admixture is heated for 20 minutes or less. In certain aspects, the admixture is heated for 15 minutes or less.

Preferably, the admixture of lutetium-177 and tetraazacyclododecane tetra-acetic acid-octreotate (DOTATATE) is heated at a temperature of about 80° C.±10° C. In certain aspects, the admixture lutetium-177 and tetraazacyclododecane tetra-acetic acid-octreotate (DOTATATE) is heated at a temperature of about 80° C.±5° C.

The method further includes cooling the admixture following heating.

In certain aspects, following the admixing, the complex of lutetium-177 and tetraazacyclododecane tetra-acetic acid-octreotate (DOTATATE) is mixed with a formulation composition. Preferably, the formulation composition is an aqueous composition. In certain aspects, the formulation composition comprises one or more ascorbate compounds such as ascorbic acid, for example in an amount of 0.5, 1, 2 or 3 mg/mL or greater.

In certain aspects, the formulation composition further includes one or more sequestering agents.

Preferably, incorporation of lutetium-177 into or with tetraazacyclododecane tetra-acetic acid-octreotate (DOTATATE) to provide ¹⁷⁷Lu-DOTATATE is greater than 98 mole percent based on the molar equivalent of lutetium-177 used in the incorporation reaction. In certain aspects, incorporation of lutetium-177 into or with tetraazacyclododecane tetra-acetic acid-octreotate (DOTATATE) to provide ¹⁷⁷Lu-DOTATATE is greater than 99 mole percent based on the molar equivalent of lutetium-177 used in the incorporation reaction.

Preferably, an acidic aqueous formulation of lutetium-177 is admixed with the tetraazacyclododecane tetra-acetic acid-octreotate (DOTATATE). In a certain embodiment, a hydrogen halide or acid halide aqueous formulation of lutetium-177 is admixed with tetraazacyclododecane tetra-acetic acid-octreotate (DOTATATE).

Preferred preparations of ¹⁷⁷Lu-DOTATATE may include one or more and preferably each of the following steps:

1. Provide lutetium-177 in a vial or other container that can serve as a reaction vessel. The lutetium-177 suitably may be present in an aqueous acidic formulation, for example, an HCl formulation.

2. Admix DOTATATE peptide with an aqueous buffer composition (Reaction Buffer) that contains one or more ascorbate compounds (e.g., sodium ascorbate and/or ascorbic acid) and preferably is substantially or completely free of other stabilizer compounds such as a gentisate compound (e.g., gentisic acid).

3. Admix the DOTATATE peptide from step 2 with the lutetium-177 composition of step 1. For example, the DOTATATE peptide composition from step 2 can be added to the vial that contains the lutetium-177.

4. The admixture of DOTATATE peptide and lutetium-177 then can be heated preferably with agitation, for example shaking with heating at 70-90° C., more typically 75-85° C. for up to 6, 7, 8, 9, 10, 11, 12, 13, 14, 15, 16, 17, 18, 19 or 20 minutes.

5. After completion of the heating treatment, the admixture from step 4 above is allowed to cool for about 0.1, 0.25, 0.5, 1, 2, 3, 4, 5, 6 or 7 minutes at or below room temperature. Cooling may be facilitated by adding an aqueous composition (Formulation Composition) containing one or more ascorbate compounds (e.g. sodium ascorbate and/or ascorbic acid) and preferably is substantially or completely free of other stabilizer compounds such as a gentisate compound (e.g., gentisic acid). The Formulation Composition added to the admixture may be at or below room temperature.

6. The admixture from step 5 then may be transferred to a vessel containing an aqueous composition (may be the same Formulation Composition as used in step 5) that comprises one or more ascorbate compounds (e.g. sodium ascorbate and/or ascorbic acid), in certain systems, preferably is substantially or completely free of other stabilizer compounds such as a gentisate compound (e.g., gentisic acid). The mixture may be sterile filtered such as through one or more 0.22 μm filter and transferred to a container such as a syringe, vial or IV bag. Desired dosages can be dispensed for administration to a patient preferably within 5, 4, or 3 days from completion of step 6.

Preferred Reaction Buffers and Formulation compositions are shown in Examples below.

### Pharmaceutical Compositions

In a further aspect, pharmaceutical compositions are provided.

Preferred pharmaceutical compositions may include an aqueous composition including 1) a complex of lutetium-177 and DOTATATE and 2) one or more ascorbate compounds. In certain aspects, such compositions are preferably substantially or completely free of other stabilizer compounds such as a gentisate compound (e.g., gentisic acid).

Preferably, the radiochemical purity of a pharmaceutical composition is at least 95% where the composition is maintained at 25° C. or less and for 3 days or more following preparation of the composition. Radiochemical purity is suitably determined by HPLC radiometric detection (i.e. radio-HPLC).

In certain preferred aspects, the pharmaceutical composition is free of unchelated lutetium-177 in an amount of not more than 2, 1.5, 1.0 or 0.5 weight % based on total weight of the pharmaceutical composition, such as may be determined by radiometric detection (including HPLC radiometric detection), where the composition is maintained at 25° C. or less and such purity levels are exhibited for 3 days or more following preparation of the composition.

In additional preferred aspects, the pharmaceutical composition is free of radiochemical impurities in an amount of not more than 5, 4, 3.5, 3, 2.5, 2, 1.5, 1 or 0.5 weight % based on total weight of the pharmaceutical composition, such as may be determined by radiometric detection (including HPLC radiometric detection), where the composition is maintained at 25° C. or less and such purity levels are exhibited for 3 days or more following preparation of the composition.

In yet still additional preferred aspects, the pharmaceutical composition is free of chemical impurities in an amount of not more than 5, 4, 3, 2, 1 or 0.5 weight % based on total weight of the pharmaceutical composition, such as may be determined by HPLC/UV analysis, where the composition is maintained at 25° C. or less and such purity levels are exhibited for 3, 4 or 5 days or more following preparation of the composition.

In yet still additional preferred aspects, the pharmaceutical composition is 1) free of unchelated lutetium-177 in an amount of not more than 2, 1.5, 1.0 or 0.5 weight % (such as may be determined by radiometric detection (including HPLC radiometric detection)); 2) free of radiochemical impurities in an amount of not more than 5, 4, 3.5, 3, 2.5, 2, 1.5, 1 or 0.5 weight % (such as may be determined by radiometric detection (including HPLC radiometric detection)); and 3) free of chemical impurities in an amount of not more than 5, 4, 3, 2, 1 or 0.5 weight % (such as may be determined by HPLC/UV analysis), with all weight % based on total weight of the pharmaceutical composition, and where the composition is maintained at 25° C. or less and such purity levels are exhibited for 3 days or more following preparation of the composition.

In certain aspects, the pharmaceutical composition is formulated for parenteral administration, such as intravenous, intramuscular, intradermal, subcutaneous, intrathecal or intraperitoneal administration. For example, the pharmaceutical composition is formulated for intravenous, intramuscular, subcutaneous or intradermal injection. In preferred aspects, the pharmaceutical composition is formulated for intravenous administration. In typical aspects, the pharmaceutical composition may be administered in a form of a pharmaceutical aqueous solution.

In certain aspects, the pharmaceutical composition is an aqueous solution, dispersion or other admixture such as for injection and comprises ¹⁷⁷Lu-DOTATATE and one or more ascorbate compounds such as sodium ascorbate and/or ascorbic acid. In certain aspects, preferably the pharmaceutical composition is substantially or completely free of other stabilizer compounds such as a gentisate compound (e.g., gentisic acid). ¹⁷⁷Lu-DOTATATE is suitably present in a concentration that it provides a volumetric radioactivity of at least 100 MBq/mL, preferably of at least 250 MBq/mL, or at least 300 or 400 MBq/mL within 1, 2, 3 or 4 days following preparation. In certain aspects, ¹⁷⁷Lu-DOTATATE is present in a concentration that it provides a volumetric radioactivity of from 100 to 1000 MBq/mL, preferably from 250 to 800 MBq/mL within 1, 2, 3 or 4 days following preparation.

In certain aspects, the one or more ascorbate compounds such as ascorbic acid may be present in a pharmaceutical composition in a total concentration of about 10 mg/mL or less, or 9 mg/mL or less, or 8 mg/mL or less, or 7 mg/mL or less, or 6 mg/mL or less, or 5 mg/mL or less, or 4 mg/mL or less.

In certain aspects, the pharmaceutical aqueous formulation has a shelf life of at least 24 hours at about 25° C. or less, at least 48 hours at about 25° C. or less, at least 72 hours at 25° C. or less, or from 24 hours to 120 hours at 25° C. or less, from 24 hours to 96 hours at 25° C. or less, from 24 hours to 84 hours at 25° C. or less, from 24 hours to 72 hours at 25° C. or less, in particular a shelf life of 72 hours at 25° C. or less. Shelf life can be assessed by radiochemical purity (as determined by radio-HPLC) remaining above 90%, 92%, 95% or 98% for the specified time period.

In certain aspects, as discussed, one or more ascorbate compounds such as ascorbic acid may be added after formation of the complex of lutetium-177 and DOTATATE, for example upon completion of heating of an admixture of lutetium-177 and DOTATATE, for example where such one or roe ascorbate compounds such as ascorbic acid are added upon temperature reduction at the conclusion of a heating step. The addition of an aqueous formulation (e.g. containing one or more ascorbate compounds such as ascorbic acid) promptly after heating is terminated can act to cool the ¹⁷⁷Lu DOTATATE reaction mixture.

In certain aspects, a pharmaceutical aqueous solution may further include a sequestering agent, for example added after formation of a complex of lutetium-177 and DOTATATE, suitably to remove uncomplexed lutetium-177. Suitable sequestering agents may include for example one or more aminopolycarboxylic acids, e.g. ethylenediaminetetraacetic acid (EDTA) and diethylenetriaminepentaacetic acid (DTPA) or a salt thereof, suitably in an amount to result in a concentration of from 0.01 to 0.50 mg/mL of the aqueous ¹⁷⁷Lu-DOTATATE composition.

In a particularly preferred aspect, ¹⁷⁷Lu-DOTATATE is provided as a sterile solution for intravenous use. A single-dose vial suitably will contain a dose of from 3.6±10% GBq to 11.1±10% GBq ¹⁷⁷Lu-DOTATATE. Four treatment cycles, represented by one injection per treatment cycle results in a cumulative dose of from 14.4±10% GBq to 44.4±10% GBq.

Preferably, the pH range of the ¹⁷⁷Lu-DOTATATE solution is 4.5 to 8.5. In a particularly preferred aspect, the pH range of the ¹⁷⁷Lu-DOTATATE solution is 5 to 7.

### Methods of Treatment

As discussed, use of ¹⁷⁷Lu-DOTATATE (including 2 above) is provided to treat cancers, including cancers originated from foregut (e.g., stomach or duodenum), hindgut (e.g., left colon or rectum), midgut (e.g., jejunum, ileum, right colon, or appendiceal), lung, and other organs (e.g., ovary, medulla, adrenal medulla, adrenal (pheochromocytoma), kidney, pituitary, thyroid or paraganglia).

In particular, ¹⁷⁷Lu-DOTATATE (including 2 above) may be used to treat neuroendocrine tumors, including to reduce neuroendocrine tumor size.

In such methods, ¹⁷⁷Lu-DOTATATE (including 2 above) can be administered to a subject such as a human in an amount effective to treat the cancer (e.g., reduction of tumor size), such as at a dose of about 3.6 GBq to about 11.1 GBq per treatment cycle, and can be suitably administered from a unit dose in a vial or a syringe or as a bulk solution in a vial or a syringe prepared from a cold-kit prepared with lutetium-177 at a local or central radiopharmacy or through cGMP central manufacturing. Total dose administered for therapy including 4 treatment cycles is about 14.4 GBq to about 40.8 GBq or 44.4 GBq.

The effective amount of the ¹⁷⁷Lu-DOTATATE radiopharmaceutical administered to a patient will generally be determined by considering the patient record. However, the effective amount suitably may be within a range of about 3.6 GBq to 11.1 GBq per dose, for example, about 3.6, 4, 4.5, 5, 5.5, 6, 6.5, 7, 7.4, 8, 8.5, 9, 9.5, 10, 10.5, 11, 11.1 GBq per dose, or any range between two of the above values. The dose can be administered from a unit dose in a vial or a syringe or as a bulk solution in a vial or a syringe prepared from a cold-kit prepared with lutetium-177 at a local or central radiopharmacy or through cGMP central manufacturing.

If necessary or desirable, the treatment may involve more than one administration of an effective amount of ¹⁷⁷Lu-DOTATATE. It is generally beneficial to repeat the administration of ¹⁷⁷Lu-DOTATATE to the subject after 7 to 56 days, such as at a 4 to 8 week interval.

In a particularly preferred protocol, the ¹⁷⁷Lu-DOTATATE dosage form is a sterile aqueous solution that is administered by intravenous injection. The dosing regimen may include multiple infusions such as 4 infusions at effective dosages such as from 3.6 GBq±10% to 11.1 GBq±10% each, administered about 4, 5, 6, 7 or 8 weeks apart.

In certain aspects, the methods include assessing neuroendocrine tumors of a patient to be somatostatin receptor positive, for example with ⁶⁸Ga-DOTATATE and a positron emission tomography scan.

### Personalized Dosing

In further aspects, dosing protocols may be utilized to provide dosing amounts for a specified patient based on one or more of the patient's characteristics.

In particular, ¹⁷⁷Lu-DOTATATE may be administered in dosage amounts based on dosimetry assessments. For example, following administration of ¹⁷⁷Lu-DOTATATE, the patient may be assessed by SPECT (e.g. 3D SPECT-CT imaging) and planar scans or other analysis to allow individualized dosimetry. Multiple scans may be performed, for example at approximately 4, 24 and 72 hours following dosing, or at a single time point or other schedule. The scans can be used to determine doses absorbed such as by tumors, kidneys and bone marrow of the patient. Based on that assessment, additional dosing of the patient can be modified, in particular either increased or decreased to enhance efficacy or safety.

Additionally, a patient's glomerular filtration rate (GFR) also may be assessed typically by a determination of creatinine (Cr) clearance.

Preferably, a patient receiving ¹⁷⁷Lu-DOTATATE will be assessed both by dosimetry and Cr clearance/GFR. By such a combined assessment, patients may be dosed with ¹⁷⁷Lu-DOTATATE at a GFR of 40 mL/min and as low as 30 ml/min.

### Combination Therapy

¹⁷⁷Lu-DOTATATE (including 2 above) suitably may be administered to a subject in conjunction or combination with one or more other therapeutic agents, particularly one or more other chemotherapeutic agents.

In one aspect, a subject may receive treatment with ¹⁷⁷Lu-DOTATATE in combination with a regime of one or more somatostatin compounds, such as octreotide (e.g. Sandostatin) or lanreotide (e.g. Somatuline Autogel).

A subject also may receive treatment with ¹⁷⁷Lu-DOTATATE in combination with a regime of one or more anticancer agents, for example capecitabine, temozolomide, steptozotocin, 5-fluroouracil, cisplatin, carboplatin, etoposide, and/or doxorubicin.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject in need of treatment as disclosed herein. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

### Packaged ¹⁷⁷Lu-DOTATATE and Kits

As discussed above, treatment kits are also provided, including cold kits where the ¹⁷⁷Lu-DOTATATE can be prepared shortly before administration such as in a medical facility, for example a hospital laboratory or radiopharmacy. In such a kit, DOTATATE may be provided in a vial or other container in lyophilized or other form separate from lutetium-177. The DOTATATE and lutetium-177 are reacted as disclosed herein at the medical facility to provide ¹⁷⁷Lu-DOTATATE which then can be promptly administered to a patient.

The kit may be a single dosage kit including ¹⁷⁷Lu-DOTATATE or pharmaceutical composition as described herein. Alternatively, the kit may be a multiple dosage kit comprising ¹⁷⁷Lu-DOTATATE or pharmaceutical composition as described herein.

In a further aspect, packaged preparations or products of ¹⁷⁷Lu-DOTATATE are also provided. A packaged preparation may comprise 1) ¹⁷⁷Lu-DOTATATE and optionally 2) instructions for using ¹⁷⁷Lu-DOTATATE for treating a cancer such as a neuroendocrine tumor. Preferably, the packaged preparation will comprise a therapeutically effective amount of ¹⁷⁷Lu-DOTATATE.

In certain exemplary packaged preparations or products, ¹⁷⁷Lu-DOTATATE can be packaged in suitable containers labeled, for example, for use as a therapy to treat a subject suffering from cancer (e.g. a neuroendocrine tumor). The containers can include ¹⁷⁷Lu-DOTATATE and one or more of a suitable stabilizer compounds as disclosed herein. A product can include a container (e.g., a vial or the like) containing ¹⁷⁷Lu-DOTATATE. In addition, an article of manufacture or kit further may include, for example, packaging materials, instructions for use, syringes, delivery devices, for treating the targeted condition, such as a neuroendocrine tumor or other cancer.

A packaged system or product may also include a legend (e.g., a printed label or insert or other medium (e.g., an audio or video file) describing the product's use). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents.

The following non-limiting examples are illustrative.

### Example 1: ¹⁷⁷Lu-DOTATATE Manufacturing

The following Table 1 describes all input materials into the manufacturing process to provide a preferred pharmaceutical composition.

TABLE 1

| | Quantity | |
|---|---|---|
| Ingredient | per batch | |
| ¹⁷⁷LuCl₃ (in 0.04N HCl) | ≤35 | GBq¹ |
| SWFI (Sterile Water for Injection) | <3 | mL |
| DOTATATE peptide | 0.35-0.85 | mg² |

Reaction Buffer

| | | |
|---|---|---|
| Ascorbic acid - 40 to 100 mg/mL | 1.6-3.9 | mL |
| NaOH - 8 to 20 mg/mL | | |

Formulation Buffer

| | | |
|---|---|---|
| Ascorbic acid - 1.8 to 4.6 mg/mL | 31.8-77.3 | mL |
| Sodium acetate trihydrate - 0.73 mg/mL | | |
| Acetic acid - 0.53 mg/mL | | |
| Sodium chloride - 7.5 mg/mL | | |
| Sodium hydroxide - 0 to 0.1 mg/mL | | |
| Diethylenetriamine pentaacetic | | |
| acid (DTPA) - 0.055 mg/mL | | |

¹At start of synthesis
²Depending on number of doses/batch

Table 2 below describes the ingredients found in the final formulated preferred drug product or pharmaceutical composition.

TABLE 2

| Ingredient | Quantity¹ | | Role in formulation |
|---|---|---|---|
| ¹⁷⁷Lu-DOTATATE | ≤0.5 | GBq/mL² | Drug substance |
| | 10 | μg/mL | |
| Ascorbic acid | 2.0-8.75 | mg/mL | Radioprotectant |
| Sodium acetate | 0.66 | mg/mL | Buffer |
| trihydrate | | | |
| Acetic acid | 0.48 | mg/mL | Buffer |
| Sterile Water for | 35-85 | mL | Diluent |
| Injection, USP | | | |
| DTPA | 0.05 | mg/mL | Chelator of free |
| | | | [Lu-177] to minimize |
| | | | physiological uptake |
| Sodium Hydroxide, | 0.36-1.00 | mg/mL | Adjustment of buffer pH |
| USP-NF | | | |
| Sodium chloride USP | 6.85 | mg/mL | Tonicity |

¹Total batch volume = 35-85 mL
³At time of calibration (TOC), defined as 07:00 ET on day of manufacture

### Example 2

The following composition was prepared in accordance with procedures set forth in Example 1 above.

The following are components and amounts thereof of a preferred aqueous composition designated herein as Composition 1:

### Composition 1

| | | |
|---|---|---|
| Lu-177 DOTATATE at ToM | 0.41 | GBq/mL |
| DOTATATE peptide | 10 | ug/mL |
| DTPA | 0.05 | mg/mL |
| Ascorbic acid | 3.52 | mg/mL |
| Sodium acetate trihydrate | 0.66 | mg/mL |
| Acetic acid | 0.48 | mg/mL |
| Sodium hydroxide | 0.65 | mg/mL |
| Sodium chloride | 6.85 | mg/mL |
| Reaction components | | |
| ¹⁷⁷LuCl₃ in SWFI | 1.6 | mL |
| Reaction buffer + peptide | 1.6 | mL |
| Total Reaction mixture Volume | 3.2 | mL |
| Product Components | | |
| Reaction mixture | 3.2 | mL |
| Formulation Buffer | 31.8 | mL |
| Final product Volume | 35 | mL |

Reaction Buffer and Radiolabeling Mixture Component Concentrations

| Component | Reaction Buffer | | Radiolabeling mixture | |
|---|---|---|---|---|
| Peptide | 219 | ug/mL | 110 | ug/mL |
| Ascorbic acid | 70 | mg/mL | 35 | mg/mL |

Formulation Buffer and Final Product Component Concentrations

| Component | Formulation Buffer | | Final product | |
|---|---|---|---|---|
| Peptide | — | | 10 | ug/mL |
| Ascorbic acid | 0.352 | mg/mL | 3.52 | mg/mL |
| Sodium acetate trihydrate | 0.726 | mg/mL | 0.66 | mg/mL |
| Acetic acid | 0.528 | mg/mL | 0.48 | mg/mL |
| Sodium chloride | 7.54 | mg/mL | 6.85 | mg/mL |
| DTPA | 0.055 | mg/mL | 0.05 | mg/mL |

The term “SWFI” herein refers to Sterile Water for Injection.

### Fabrication Process

In a Step 1 described in this section takes place in a Grade A biological safety cabinet. All subsequent steps described in this section take place within a Grade A shielded isolator. All solutions, including Reaction Buffer and Formulation Buffer, are prepared in a Grade C clean room. In summary, in the incorporation reaction, the DOTATATE peptide is mixed with reaction buffer and ¹⁷⁷LuCl₃, followed by heating and shaking to form the drug complex substance.

The heater/shaker undergoes installation, operation and performance qualification prior to use. Qualification has confirmed that the required temperature and shaking conditions for the incorporation reaction (radiolabeling) can be maintained for the specified time period, as described in Step 4 below.

The amount of DOTATATE peptide used (Step 2) is based on the total amount of ¹⁷⁷Lu-DOTATATE activity (itself based on the number and timing of patient doses) and the volume required to yield a radioactive concentration of ≤0.96 GBq/mL at time of calibration (TOC). The TOC is defined as 07:00 ET on the day of manufacture (DOM).

1. Depending on the amount of ¹⁷⁷Lu starting activity required, the appropriate volume of 0.04 N HCl is added to the supplier vial containing ¹⁷⁷LuCl₃ in 0.04 N HCl to bring the total volume to
a. 1000 μL (if 160-320 μg of DOTATATE peptide are used)
b. 2000 μL (if 480-640 μg of DOTATATE peptide are used)

2. 1-4×750 μL volume(s) of DOTATATE peptide are prepared in reaction buffer. The solution is gently agitated to ensure complete dissolution of the peptide. Each 750 μL volume of DOTATATE peptide solution contains 200 μg DOTATATE.

3. If only 1× or 3×750 μL aliquots of DOTATATE solution are required, an additional 1×600 μL volume of reaction buffer is prepared.

4. The reaction solution is prepared by adding 600 μL volumes of DOTATATE solution from Step 2 (and if applicable reaction buffer from Step 3) to the supplier vial from Step 1 (containing diluted ¹⁷⁷LuCl₃). The total reaction solution volume is 2.2 mL (160-320 μg of peptide) or 4.4 mL (480-640 μg of peptide).

5. Using the qualified and pre-heated heater/shaker, the reaction solution is heated at 80±5° C. for 15±1 min with shaking at 350 rpm.

6. The reaction solution vial containing the drug substance is allowed to cool at room temperature for at least 5 min.

Subsequent steps consist of transferring the drug substance (in reaction buffer) through a tandem 2×0.22 μm sterile filter chain, to a 100 mL sterile bulk vial containing formulation buffer, and aseptic dispensing of the final drug product into the final container closure. These steps are accomplished using a dispensing apparatus consisting of a series of stopcocks to which various tubing, syringes, vials and needles are attached to facilitate transfer and dispensing of the drug product. Steps 7-10 take place within a Grade A shielded isolator unit.

7. The ¹⁷⁷Lu-DOTATATE drug substance is immediately transferred from the reaction vial, through a tandem 2×0.22 μm sterile filter chain, to a 100 mL bulk vial that contains (post-reaction vial rinsing) 6.95-115.6 mL of formulation buffer. This step forms the drug product and represents end of synthesis (EOS).

8. Samples are removed from this vial for sterility and QC testing.

9. Patient doses are dispensed into final 30 mL container closures.

The total synthesis time, from Step 3 to Step 7 described above, is approximately 120 min.

### Example 3: Treatment Protocol

A human patient with neuroendocrine tumors is selected for treatment after their tumors have been shown to be somatostatin receptor positive with ⁶⁸Ga-DOTATATE and a positron emission tomography scan.

¹⁷⁷Lu-DOTATATE in a sterile aqueous solution as designated as Composition 1 in Example 2 above is administered by intravenous injection to the patient. Single-dose vials are used that contain from 3.6±10% GBq to 11.1±10% GBq ¹⁷⁷Lu-DOTATATE. The ¹⁷⁷Lu-DOTATATE is administered over four treatment cycles, with one injection per treatment cycle that provides a cumulative dose of from 14.4 GBq to 44.4 GBq.

### INCORPORATION BY REFERENCE

The entire contents of all patents, published patent applications and other disclosures cited herein are hereby expressly incorporated herein in their entireties by reference.

While this invention has been particularly shown and described with references to preferred embodiments thereof, it will be understood by those skilled in the art that various changes in form and details may be made therein without departing from the scope of the invention encompassed by the appended claims.

1. A pharmaceutical composition comprising:
(a) a complex of lutetium-177 and tetraazacyclododecane tetra-acetic acid-octreotate (DOTATATE); and
(b) one or more ascorbate compounds, wherein the composition is substantially or completely free of stabilizer compounds other than the one or more ascorbate compounds.

2. (canceled)

3. The pharmaceutical composition of claim 1 wherein the one or more ascorbate compounds are present in the composition in an amount of 10 mg/mL or less.

4-6. (canceled)

7. The pharmaceutical composition of claim 3 wherein the one or more ascorbate compounds are present in the composition in an amount of between 2.5 and 4.5 mg/mL.

8. The composition of claim 1 wherein the complex of lutetium-177 and tetraazacyclododecane tetra-acetic acid-octreotate (DOTATATE) is present in an amount of at least 5 mCi/mL.

9. (canceled)

10. (canceled)

11. The pharmaceutical composition of claim 1 wherein the radiochemical purity of the composition is 95% or greater for 3 days by HPLC analysis.

12-18. (canceled)

19. The method of claim 66 wherein the admixture is heated for 60 minutes or less.

20-24. (canceled)

25. The method of claim 66 wherein the admixture is heated at about 80° C.±10° C.

26-30. (canceled)

31. The method of claim 66 wherein the formulation composition comprises the one or more ascorbate compounds in an amount of 1 mg/mL or greater.

32. (canceled)

33. The method of claim 66 wherein the formulation composition further comprises one or more sequestering agents.

34. The method of claim 66 wherein the reaction of lutetium-177 with tetraazacyclododecane tetra-acetic acid-octreotate (DOTATATE) provides ¹⁷⁷Lu-DOTATATE in greater than 98 mole percent.

35-40. (canceled)

41. The method of claim 67 wherein the one or more ascorbate compounds are present in the pharmaceutical composition in an amount of 10 mg/mL or less.

42. (canceled)

43. The method of claim 41 wherein the one or more ascorbate compounds are present in the pharmaceutical composition in an amount of 6 mg/mL or less.

44-50. (canceled)

51. The pharmaceutical composition of claim 1 wherein the radiochemical purity of the composition is 95% or greater for 3 days or more at 25° C.

52-56. (canceled)

57. The pharmaceutical composition of claim 1 wherein the composition as a pH of 4.0 to 6.0.

58. A single dosage kit comprising the pharmaceutical composition of claim 1.

59. A multiple dosage kit comprising the pharmaceutical composition of claim 1.

60. A sealed container comprising or the pharmaceutical composition of claim 1.

61. (canceled)

62. The method of claim 67 wherein the subject is suffering from a neuroendocrine tumor.

63. The method of claim 62 wherein the neuroendocrine tumor originates from foregut, hindgut, midgut, lung, ovary, medulla, adrenal medulla, adrenal, kidney, pituitary, thyroid or paraganglia.

64. (canceled)

65. (canceled)

66. A method for preparing the pharmaceutical composition of claim 1, comprising the steps of:
a) admixing lutetium-177 and tetraazacyclododecane tetra-acetic acid-octreotate (DOTATATE) in aqueous solution in the presence of one or more ascorbate compounds in a concentration of 40 mg/mL or less, in the substantial or complete absence of a second stabilizing agent, and
b) heating the admixture, thereby forming an aqueous solution comprising a complex of lutetium-177 and tetraazacyclododecane tetra-acetic acid-octreotate (DOTATATE);
c) mixing the aqueous solution of step b) with an aqueous formulation composition comprising one or more ascorbate compounds in the substantial or complete absence of a second stabilizing agent.

67. A method of treating cancer in a subject in need thereof, comprising administering to the subject a therapeutically effective amount of the pharmaceutical composition of claim 1.
